(19) United States
(12) Patent Application Publication
(10) Pub. No.: US 2016/0220757 A1
(43) Pub. Date: Aug. 4, 2016

(54) NEEDLE-FREE SUBCUTANEOUS APPLICATION OF PROTEINS

(71) Applicant: LTS LOHMANN THERAPIE-SYSTEME AG, Andernach (DE)

(72) Inventors: Stefan HENKE, Kirchen (DE); Heiko SPILGIES, Koblenz (DE); Karsten HEUSER, Bad Breisig (DE); Sebastian SCHERR, Neuhäusel (DE); Uwe WORTMANN, Marburg (DE); Andreas HENNING, Koblenz (DE); Claudia HAMMES, Andernach (DE); Rolf PRACHT, Höhr-Grenzhausen (DE); Jörg BENDER, Köln (DE)

(21) Appl. No.: 14/916,831

(22) PCT Filed: Sep. 9, 2014
(86) PCT No.: PCT/EP2014/069229

§ 371 Date: Mar. 4, 2016

(30) Foreign Application Priority Data
Sep. 9, 2013 (EP) 13183621.5

(51) Int. Cl. A61M 5/30

(52) U.S. Cl. CPC A61M 5/30; A61M 2202/07

(57) ABSTRACT

The invention relates to the needle-free subcutaneous administration of proteins to humans and animals by means of a protein delivery device comprising a unit made of a nozzle, a chamber, a piston, an actuating device, and a drive, and a corresponding process and its use.

The invention relates to the needle-free subcutaneous administration of proteins to humans and animals by means of a protein delivery device comprising a unit made of a nozzle, a chamber, a piston, an actuating device, and a drive, and a corresponding process and its use.

The administration of proteins to humans and animals presents a special challenge, on proteins are usually administered via the subcutaneous route using non-needle-free means (e.g., a syringe or something similar). In particular, when therapeutic proteins are administered via a transdermal route they must pass through the skin's own proteins. However proteins, especially recombinant proteins, have assumed growing significance as drugs. In particular, these proteins concern such active ingredients as growth factors, antibodies, hormones, enzymes, inhibitors/receptor antagonists, clotting factors, and cytokines.

Active ingredients based on proteins (especially recombinant proteins) are routinely used today for the following indications or reasons for treatment:

antithrombotics, asthma, respiratory infections, antigens, anemia (epoetin), blood disease, diabetes (insulin), disorders of fertility , hepatitis B/C (PEG-interferon alpha-2a/2b), bone fractures, cancer, macular degeneration (ARMD), cystic fibrosis (dornase alfa), multiple sclerosis (natalizumab), osteoporosis (teriparatide), paroxysmal nocturnal hemoglobinuria, rheumatism (infliximab/adalimumab), mucositis (palifermin), psoriasis, sepsis (drotrecogin alfa), metabolic diseases, transplantation (basiliximab), disorders of growth/acromegaly (somatropin/pegvisomant), disorders of growth/dwarfism (somatropin), and wound healing (becaplermin).

In Europe, more than 150 genetically engineered drugs, especially recombinant proteins, are approved at the present time.

so However it is significant that when recombinant proteins are produced with the hosts used, such as bacteria (e.g., Escherichia coil), such proteins are not, in contrast to native protein, processed, e.g., they are not glycosylated. Consequently, these recombinant proteins are usually not structurally identical with their authentic counterpart, but rather only functionally identical.

A largely similar glycosylation pattern is obtained for proteins that are produced by means of well-established cell lines such as baby hamster kidney cells (BHK cells), Chinese hamster ovary cells (CHO cells), or human fibroblasts.

This means that there are strong regulatory requirements, for example, on therapeutic proteins, both in their production and also in their form of dosage or administration.

Furthermore, it must be ensured that especially the function-determining tertiary and quaternary structure of a protein is preserved, and not affected by the administration, or that it remains largely preserved.

Therefore, in order to preserve the function of proteins, especially recombinant or therapeutic proteins, also with regard to sufficient drug efficacy and safety, it is essential to provide a new form of administration that will maintain the high quality of the drug that is used.

The prior art describes the non-needle-free subcutaneous administration of proteins to be administered.

There continues to be a pressing need for new innovative administration systems, so that, e.g., the dose or the pharmacokinetics of an administration can advantageously be optimized (e.g., reproducibility, efficacy, etc.).

Now it was surprisingly discovered that needle-free subcutaneous administration of a protein to humans and animals by means of a protein delivery device comprising a unit made of a nozzle, a chamber, a piston, an actuating device, and a drive is excellently suitable for this purpose.

In an especially preferred embodiment, the needle-free subcutaneous administration is done perpendicular to the skin's surface, rather than tangential to it.

Therefore, the invention relates to a protein delivery device comprising a unit made of a nozzle, a chamber, a piston, an actuating device, and a drive for use in the needle-free subcutaneous administration (of a protein) to humans or animals.

In another preferred embodiment, the device is designed as a disposable system, so that only a single use is possible. Consequently, the protein to be administered is already provided in the chamber. To accomplish this, the chamber can be filled with the protein and routinely prepared in the device (also called an applicator), e.g., by means of a usual snap device.

The chamber can contain the protein to be administered in the form of a protein solution or a protein melt.

For example, the protein solution or protein melt can contain an aqueous buffer solution and other customary additives and excipients.

Such a protein solution can also comprise a formulation consisting of protein, a liquid medium, and polysaccharides, as are necessary, for example, for therapeutic proteins, especially vaccines.

It is known that such systems, which are polymer fluids, can be hydrodynamically described as non-Newtonian fluids, and have special flow behavior that is the subject matter of the science of rheology (H. Pleiner, M. Liu, H. R. Brand, Rheol. Acta 39, 560 (2000)). Therefore, depending on the protein concentration, the solution can exhibit special theological behaviors (dilatancy, rheopexy, turbulence, etc.). Macroscopically, as the protein concentration increases, the solution's viscosity increases (its fluidity decreases), in which shear stress plays a role. Finally, these shear stresses in the molecular plane of a protein should be attributed to the primary, secondary, tertiary, and quaternary structure as a function of the protein concentration at a given pressure and temperature (folding, network structure, coil, etc., which affect, e.g., the Huggins coefficient, etc.).

It was also surprisingly discovered that a cylindrical chamber having, on its end, a radial taper that opens into a nozzle allows optimized shear thinning as a function of the shear rate of the just described protein solutions, which are a polymer fluid. This allows the important therapeutic proteins to maintain, to a large extent, their efficacy, and for the administration of the proteins to be qualitatively gentle. Consequently, the invention made it possible to achieve an optimized chamber for proteins for their needle-free subcutaneous administration.

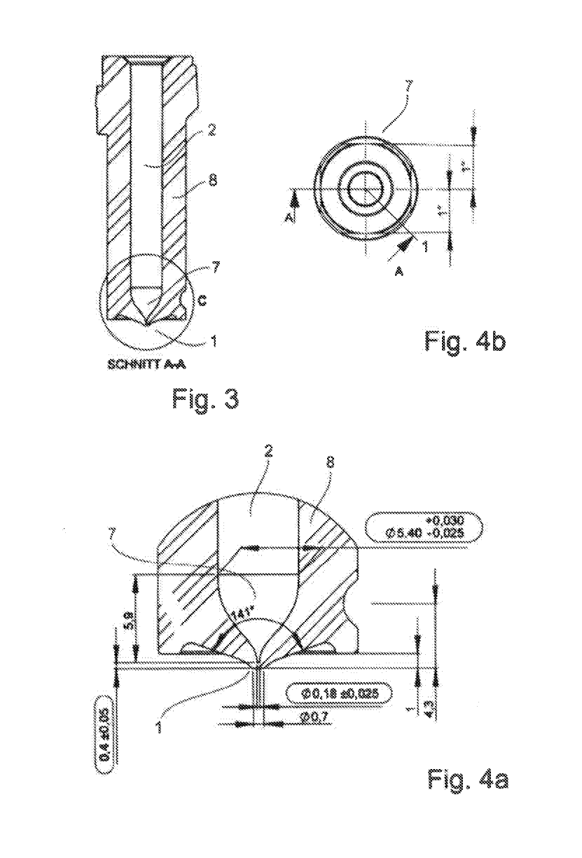

FIGS. 3, 4a, and 4b show an example of such a cylindrical chamber having, on its end, a radial taper that opens into a nozzle. In one preferred embodiment, the taper is funnel-shaped. For example, a cylindrical chamber having a diameter of 4 to 7 mm can have a taper or funnel that is 4 to 7 mm long (see FIG. 4a).

Therefore, the invention relates to a protein delivery device comprising a unit made of a nozzle, a chamber, a piston, an actuating device, and a drive, also for use in the needle-free subcutaneous administration (of a protein) to humans or animals, the device comprising a cylindrical chamber having, on its end, a radial taper that opens into a nozzle.

In another preferred embodiment, the chamber or chamber walls consist of an inert material. preferably a plastic, in particular a thermoplast with good thermoplastic flow properties, high rigidity, strength, and hardness, which produce small frictional forces for the protein, such as, e.g., cycloolefin copolymers (COC), especially Topas®. In addition, COC advantageously have high biocompatibility with respect to proteins.

The previously mentioned inventive measures (the geometry and material of the chamber) advantageously also prevent the chamber from bursting during use or administration.

Therefore, another embodiment of the invention relates to a chamber containing a protein, in particular a protein solution, the chamber consisting of or being made from a plastic, in particular a thermoplast, preferably cycloolefin copolymers (COC), especially preferably Topas®. The chamber can be produced by means of an injection molding process, for example.

In another preferred embodiment, the device is immediately prepared for needleless subcutaneous administration by removing a cap.

The inventors were able to show that needleless subcutaneous administration produces surprisingly high plasma values of the administered protein. An essential aspect of the invention is that the administered protein preserves the functionality responsible for its specific effect, making the inventive administration most highly suitable for drugs based on a protein, for example. This allows reproducible dosage, and increases patient safety. In addition, it ensures improved pharmacokinetics.

In another preferred embodiment, the drive consists of a spring drive, such as described by the applicant, e.g., in DE 10 2008 063 519 A1, DE 10 2007 004 211 A1, DE 10 2007 018 868 A1, or DE 10 2007 032 464 A1, or a gas drive, such as described, e.g., in EP 1 125 593 61 or EP 1 243 281 B1, or a pyrotechnic drive, such as described in EP 1 292 344 B1.

In another preferred embodiment, the front closed end of the chamber has at least one or more nozzles, or even multi-hole systems, as described, e.g., in DE 20 2008 017 814 U1. A suitable nozzle can be implemented, for example, by a hollow body with an entrance and an exit. The diameter can be, e.g., 0.1 mm to 1 mm.

The chamber volume can preferably be from 0.1 mL to about 2.0 mL, taking into consideration different inside diameters of the chamber.

The chamber is suitable to hold a protein to be administered.

In the context of this invention, a “protein” is understood to be a polypeptide, which might be chemically modified, for example by glycosylation, alkylation, etc. The protein can also be a drug or therapeutic agent. It is further preferred for the protein to be a recombinant protein, including an antibody, in particular a monoclonal or polyclonal antibody, an antigen, or a protein that has one or more epitopes. The proteins can also be defined by their biochemical function, such as, but not limited to, growth factors, antibodies, hormones, enzymes, inhibitors/receptor antagonists, clotting factors, vaccines, and cytokines, in either a protein solution or a protein melt (corresponding to a polymer melt). It is also possible for one or more of the same or different proteins to be present. It is also preferred for the proteins to have more than 50 amino acids, especially more than 100 amino acids and/or a molecular mass greater than 1 kDa, especially greater than 10 kDa.

The term “needle-free” means that it is not necessary to insert a needle into the tissue (skin), but rather the inventive device is suitable for injection, however without making use of a needle in the broadest sense. The term “needleless” injection can be used as a synonym.

The term “needle-free subcutaneous administration” means that a protein is administered via the parenteral or transdermal route, this administration affecting the tissue under the skin. This hypodermis (subcutaneous tissue or subcutis) consists essentially of the connective tissue and adipose tissue lying directly under the skin. According to the invention, it is essential that the administered protein passes into the blood stream and that it can be detected in the plasma.

The invention also relates to a process for needle-free subcutaneous administration of a protein to humans or animals, wherein a.) a protein delivery device comprising a unit made of a nozzle, a chamber, a piston, an actuating device, and a drive is placed near or onto a skin surface; b.) this protein delivery device is optionally oriented perpendicular to the skin surface; and c.) after the actuating device is triggered, this protein delivery device is held on the skin surface for at least 10 seconds. The process can be further designed according to one of the prototype embodiments of an inventive device.

The invention also relates to the use of a protein delivery device comprising a unit made of a nozzle, a chamber, a piston, an actuating device, and a drive for the needle-free subcutaneous administration of a protein to humans or animals. This use can be further designed according to one of the prototype embodiments of an inventive device or an inventive process.

Moreover, the invention relates to means containing proteins for use in the needle-free subcutaneous administration of a protein to humans or animals comprising a device consisting of a unit made of a nozzle, a chamber, a piston, an actuating device, and a drive. The means can be further designed according to one of the prototype embodiments of an inventive device or an inventive process.

The following examples and figures serve to explain the invention in detail, without, however, limiting the invention to them.

### EXAMPLE 1

The needle-free administration system (also called an applicator) is filled with 0.5 mL of adalimumab (Humira® 40 mg/0.8 mL). Each administration delivers 25 mg of adalimumab subcutaneously. Pigs are used as an approved animal model.

Blood samples (200 μL EDTA plasma samples) are taken at time intervals and centrifuged at 2,500 g for 15 minutes at room temperature. The data is pharmacokinetically analyzed using WinNonlin 7 (Pharsight Corp., Mountain View, Calif., USA) and the AUC values are extrapolated and determined (linear trapezoidal method).

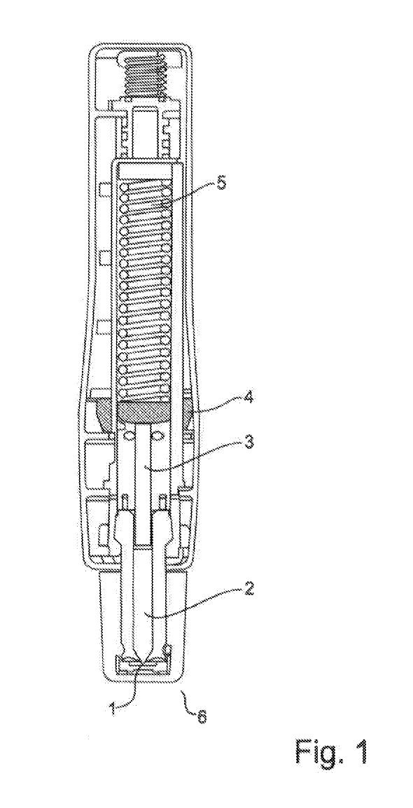

FIG. 1 shows an example of an inventive device consisting of a unit made of a nozzle (1), a chamber (2), a piston (3), an actuating device (4), and a drive (5), along with a removable cap (6).

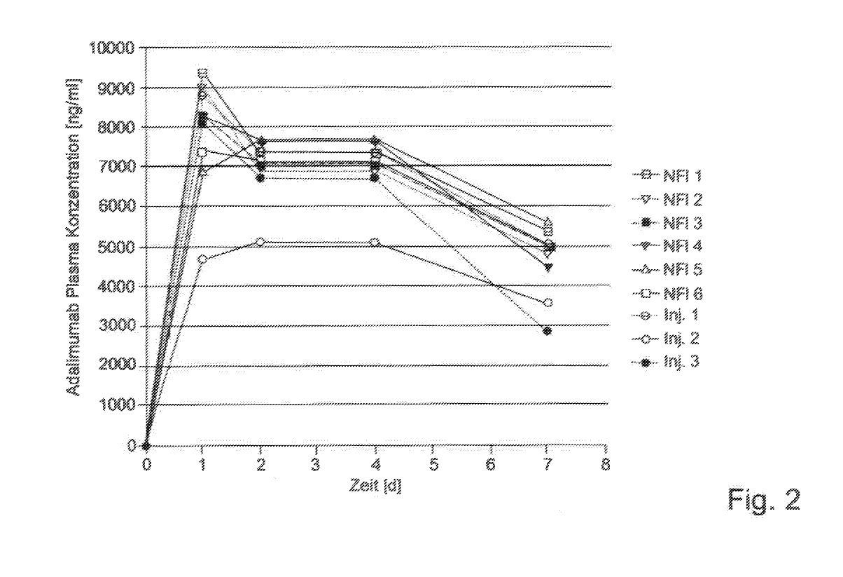

FIG. 2 shows a plot of the plasma concentration (ng/mL) of adalimumab from example 1 vs. time in days (d) comparing the needle-free subcutaneous administration (line marked by triangles and labeled “NFI”) with non-needle-free subcutaneous administration (rectangles, “Inj.”), starting from the same quantities/dosage. The surprisingly high values of the plasma concentration following needle-free subcutaneous administration can clearly be seen.

FIG. 3 is a longitudinal section through a detail showing a cylindrical chamber with a radial taper (7) on its end opening into a nozzle.

FIG. 4a is a longitudinal section through the detail in FIG. 3 showing a cylindrical chamber with a radial taper (7) on its end opening into a nozzle.

FIG. 4b shows a cross section of a cylindrical chamber with a radial taper (7) on its end opening into a nozzle.

### LIST OF REFERENCE NUMBERS

FIG. 1

1 Nozzle

2 Chamber

3 Piston

4 Actuating device

5 Drive (here a spring drive)

6 Removable cap

7 Radial taper on the end of the chamber opening into a nozzle

8 Chamber wall

1.-18. (canceled)

19. A protein delivery device comprising a unit made of a nozzle, a chamber, a piston, an actuating device, and a drive for use in the needle-free subcutaneous administration of a protein to humans or animals.

20. The device described in claim 19, wherein the chamber contains a protein.

21. The device described in claim 19, wherein the device has a cylindrical chamber with a radial taper on its end that opens into a nozzle.

22. The device described in claim 21, wherein the taper is funnel-shaped and the length of the taper is 4 to 7 mm.

23. The device described in claim 19, wherein the chamber walls of the chamber consist of a plastic, in particular a thermoplast.

24. The device described in claim 19, wherein the chamber walls of the chamber consist of a plastic.

25. The device described in claim 19, wherein the chamber walls of the chamber consist of a cycloolefin copolymer (COC).

26. The device described in claim 19, wherein the needle-free subcutaneous administration is done perpendicular to the skin surface.

27. The device described in claim 19, wherein the drive is a spring drive, a gas drive, or a pyrotechnic drive.

28. The device described in claim 19, wherein the protein, is drugs, therapeutic agents, recombinant protein, antibodies, antigens, growth factors, hormones, enzymes, inhibitors/receptor antagonists, clotting factors, vaccines and/or cytokines, protein solution, protein melt.

29. A protein delivery device comprising a unit made of a nozzle, a chamber, a piston, an actuating device, and a drive, wherein the device has a cylindrical chamber with a radial taper on its end that opens into a nozzle, and/or the device has chamber walls of the chamber made of a plastic.

30. Means containing proteins and the device as claimed in claim 19.

31. The means as claimed in claim 30 wherein the device is consisting of a unit made of a nozzle, a chamber, a piston, an actuating device, and a drive for use in the needle-free subcutaneous administration of a protein to humans or animals.

32. The means as claimed in claim 30, wherein the device has a cylindrical chamber with a radial taper on its end that opens into a nozzle, and/or the device has chamber walls of the chamber made of a plastic.

33. The means as claimed in claim 30, wherein the device has a cylindrical chamber with a radial taper on its end that opens into a nozzle, wherein the taper is funnel-shaped and the length of the taper is 4 to 7 mm.

34. The means as claimed in claim 30, wherein the device has a cylindrical chamber with a radial taper on its end that opens into a nozzle, and the device has chamber walls of the chamber made of a plastic.

35. The means as claimed in claim 30, wherein the protein is adalimumab.

36. The device as claimed in claim 19, wherein the device contains a protein and the protein is adalimumab.

37. A disposable system containing the device described in claim 19, the device for needle-free subcutaneous administration being prepared by means of a removable cap.

38. A process for needle-free subcutaneous administration of a protein to humans or animals, wherein
a.) a protein delivery device comprising a unit made of a nozzle, a chamber, a piston, an actuating device, and a drive is placed near or onto a skin surface;
b.) said protein delivery device is optionally oriented perpendicular to the skin surface; and
c.) after the actuating device is triggered, said protein delivery device is held on the skin surface for at least 10 seconds.
